(19) United States
(12) Patent Application Publication
(10) Pub. No.: US 2006/0257319 A1
(43) Pub. Date: Nov. 16, 2006

(54) Method for in vivo determination on tested animals of the efficient concentration of deuterium depleted water for cancer therapy

(76) Inventors: Nicolae Manolescu, Bucharest (RO); Ion Balanescu, Bucharest (RO); Serban Constantin Valeca, Pitesti (RO); Rodin Traicu, Drobeta Tr. Severin (RO); Dumitru Marculescu, Mehedinti (RO); Petru Niculita, Bucharest (RO); Joan Stefanescu, Ramnicu (RO); Ioan Terbea, Bucharest (RO); Victoria Moraru, Bucharest (RO); Virgilliu Comisel, Bucharest (RO); Corneliu Mateescu, Bucharest (RO); Ioan Encut, Bucharest (RO); Marieta Panait, Bucharest (RO); Daniela Begu, Bucharest (RO); Sabin Cinca, Bucharest (RO); Maria-Iuliana Gruia, Bucharest (RO); Emilia Balint, Ilfov (RO); Aneta Pop, Bucharest (RO)

Correspondence Address: HERSHKOVITZ & ASSOCIATES, 2845 DUKE STREET, ALEXANDRIA, VA, 22314, US

(21) Appl. No.: 10/567,842

(22) PCT Filed: Jul. 20, 2004
(86) PCT No.: PCT/RO04/00012

§ 371 Date: Feb. 10, 2006

(30) Foreign Application Priority Data
Aug. 13, 2003 (RO) A 2003 00685

(51) Int. Cl. A61K 49/00

(52) U.S. Cl. 424/9.2

(57) ABSTRACT

Deuterium Depleted Water is administered to tested animals before and after tumoral grafting with animal grafts in certain concentrations over a period of time, viability is determined for the tumor cells to be grafted, grafting of the animals in the experimental group and the control animals is conducted, continuously and long-term administering, by diet, of Deuterium Depleted Water, in certain concentrations is performed, and determination is made of efficient concentration of Deuterium Depleted Water for tested surviving animals depending on certain characteristics.

The present invention refers to a method for in vivo determination, on tested animals, of an efficient concentration of Deuterium Depleted Water for cancer cure—method that could be embodied in experimental oncology.

There are known methods and installations for Deuterium Depleted Water obtaining from natural water or from Heavy Water manufacturing process (Patent No. RO 112422; Application for Patent No: FR 2 552 324; International Application No. PCT WO 96/33129; Patent No RU 2 182 562).

Also, Deuterium Depleted Water properties are well known as regarding amelioration or curing of various diseases including cancer, when this kind of water is administered to patients, as it is, or as prepared pharmaceutical products, or as cosmetics (U.S. Pat. No. 5,788,953, WO 96/03996, WO 95/18545).

From background of the invention, it is understood that an experimental method (U.S. Pat. No. 5,788,953) for in vivo determination of needed concentration of Deuterium Depleted Water for cancer therapy is known, but this method shows numerous disadvantages. Thus, within the described method, human tumors are used (prostate tumor, breast tumor, etc.) and are grafted on immunosuppressed animals. This xenotransplant (i.e. human tumor grafted on animals) has been obtained on inbred lines of mice, that is the CBA/Ca pure line, with the animals being prior immunosuppressed (WO95/18545 pag.3).

But, it is known that regarding malign human tumor transplanted to animals, there is a major risk of rejection (Billingham, R. E. et al., 1953, Nature, 172, 603; Miles, C. P., 1965, J.Natl. Cancer Inst. 34, 103; Comisel V. et al, 2001, Romanian Journal of Comparative Oncology, 4, 295).

It is already well known that xenotransplant of cells, tissues and organs is extremely difficult to be done, almost impossible, since the xenotransplanted part is rejected by the host as the time goes by. Also, the malign tumoral grafts are rejected in xenogenic system. Generally, in respect of tumor xenotransptant, and malign tumor particularly, to the tested animals, there are numerous published papers presenting and discussing the conditions favorable for the success of the tumoral xenotransplant, such as:

a) use of a special techniques for tumor cell inoculation (intra-embryonic inoculation; intra-cranial inoculation at new-borne hamster; intra-testicular inoculation; inoculation in anterior chamber of eye; under renal capsule, in cheek pouch of the hamster etc.). Tumor xenotransplant in so-called privileged spots has shown that the percentage of positive grafts is not significant and it is also variable, and therefore, it cannot provide a constant and reproducible experimental model (Comisel V. et al, 2001, Romanian Journal of Comparative Oncology, 4, 295);

b) use of animals having congenital or developed immunodeficiency (“nude” homozygous mice that are congenital athymic being characterized by a deficiency of cellular immediated immune response, surgeon thymectomy of new-borne animals);

c) immune reactivity inhibition to induce specific tolerance to xenotransplant by various methods, such as: irradiation with non-lethal dose and under antibiotics protection; blocking of immune system by intra-vein inoculation with large dose of colloidal suspensions; administering of corticoid over renal steroids or anti-lymphocytar serums or immunosuppressive drugs (cyclophosphamide, cytostar, etc.), or cyclosporine. The known means for the generation of the immunosuppressive condition on animals to be subject to malign tumor xenotransplant have proven to have adverse effects on healthy condition of immunosuppressed animals, and this condition is affecting the results of experimental tests (Comisel V. et al, 2001, Romanian Journal of Comparative Oncology, 4, 295).

Also, the embodiment of a treatment of immunology suppression before the transplant performance, after the transplant, during the experiment of efficient concentration of deuterium depleted water establishing, leads to the finding that the dose effect wouldn't be the real one, since the immunologically suppressed animal has completely different responses compared to a normal animal. It does not respond or, from the immunology point of view, its response level is very low.

Another important aspect related to xerotransplant is the fact that immunosuppressed animals recover their capacity of rejecting the normal tissue graft or the malign tumor tissue after a period of time of 4-6 weeks, no matter how the immunosuppressing process has been induced.

On the experiments described in U.S. Pat. No. 5,855,921, it is appreciated that the human malign tumors transplanted to animals and developed into them, over a period of time of administering Deuterium Depleted Water treatment, that malign tumors have regressed and then, they have been rejected due to this treatment. But, in this case, the rejection could be a result of a normal immunological reaction, i.e. the host against tumor graft, as we have described above.

Also, the statement that malign tumors transplanted to animals and then treated with Deuterium Depleted Water wouldn't grow into metastasis could not be taken into consideration as being a result of Deuterium Depleted Water administering since, as we have already demonstrated above, the human malign tumors experimentally transplanted do not grow into a metastasis or they do, but very rarely.

In U.S. Pat. No. 5,855,921, Deuterium Depleted Water concentration administered during the experiment, are not constant, so as a single efficient concentration could not be established. Thus, during the experiment, 30 ppm Deuterium content water is administered, at the beginning, over a period of 3 weeks, and then, on the same group of animals, 110-120 ppm Deuterium content water is administered until the end of the experiment. This is the way Deuterium content range is made up, according to the U.S. Pat. No. 5,855,921.

Taking into consideration the afore mentioned, we can conclude that the experiments in the background of the invention fathered by Mr. Gabor Somlayi are not convincingly because, for the applied xerotransplat, the results are affected by the conditions under which the experiments have been conducted: the way of concentration administering over a too large range of concentrations respectively, and the use of human tumor on priory immunosuppressed animals. Also, from the experiments showed in U.S. Pat. No. 5,855,921 total duration for cancer therapy is not clearly demonstrated, respectively, the duration and concentration for the maximum effects are not indicated.

Technical issue the invention is solving is the establishing of a method for experimental determination in vivo of an efficient Deuterium content in water, in order to obtain optimum results in cancer therapy on rats.

According to the invention, the method consist in Deuterium Depleted Water administering before and after tumor grafting on animals, following the stages below:
A) Deuterium Depleted Water administering to Wistar outbred rats by diet, with concentration less than 100 ppm, over a period of 60 days, simultaneously to dieting a control group of animals with water having 150 ppm content of Deuterium (tap water), over the same period of time;
B) Viability determination for the tumor cells to be grafted, using tripan blue;
C) Grafting of the animals in the experimental group and the control animals in the 60th day, subcutaneous, with 1×10⁷ malign tumor cells in 0,5 ml normal saline solution of 256 Walker sarcoma (the solid tumor) and T8 Guérin lymphotropic epitelioma (solid tumor), both of them having cells with a viability over 98%;
D) Continuously and long-term administering, by diet, of Deuterium Depleted Water, with concentration less than 100 ppm deuterium, period over which the followings are to be done:
   a) Starting with the 4-th post-graft day the tumor nodules measurement and examination is performed on each 2-3 days;
   b) Monitoring of animals physiological condition by weekly weighing, monitoring their food and water consumption, notifying the toxic condition occurrence;
   c) After 60 days, when all the animals in control group aredead, preferable between the 160th and 200th day after graft, the effect produced by administering of established concentration of Deuterium Depleted Water is observe don the surviving animals homeostasis from experimental groups, respectively the way how humoral immune system and cellular immune system of these animals has been influenced, by performing of a series of examination on immunological condition of the animals, namely: leucocytes formula test to establish lymphocytes and blastic cells levels; hematopoietic marrow tests to establish the plasmocytes and NK-K cells levels; and
E) Determination of efficient concentration of Deuterium Depleted Water for tested surviving animals depending on new homeostasis occurrence, and on the results obtained related to tumoral regression, as well as to cancer curing.

The advantages of this method as per invention are the following:
this is a method applying allogenic tumoral graft that could rather enable an extrapolation of findings to human bodies than the findings obtained by other Patent authors using singeneic animals;
it allows exact determination of the efficient Deuterium concentration;
the results of method embodiment are accurate and reproducible, the tumors grafted in allogeneic system having a 100% percentage of tumor catching, without spontaneous tumor regressions;
animal immunological suppression is not performed that allow us to eliminate the possibility of fake positive results;
there is no arbitrary factor introduced in establishing the Deuterium concentration;
well known experimental tumors are used for screening, which are currently used to evaluate the cytostatics effects.

Herein below there is an example for method embodiment as per invention.

As per invention, the method consists in Deuterium Depleted Water administering to animals to be tested (rats) of Deuterium Depleted Water, before and after tumor graft with animal grafts.

The method includes the following stages:

A) Administering of Deuterium Depleted Water Before Tumor Graft
Approximately 800 animals to be tested are selected, outbred Wistar rats, respectively, males and females, having a weight of 120±20 gr, on a good physiological condition observed after a clinical examination
The animals to be tested were distributed as 7-8 per cage (males separated from females). Deuterium Depleted Water is administered to three groups of rats, having a concentration of 25 ppm; 60 ppm and 100 ppm, over a period of 60 days, simultaneously with administering to control animals a 150 ppm Deuterium water over the same period of time (tap water).

B) Determination of Tumor Cells Viability that are Going to be Grafted
Before graft performance the viability of tumor cells to be grafted is determined with tripan blue. Viewing this determination, tumoral cells are collected on a microscope blade; the cells are obtained as per known procedures, from alogeneic tumor in 0.5 cc natural saline solution and 1-2 drops of tripan blue are added. The blue colored cells are dead cells. The calculation of cells viability is done by dead cells counting out from 1000 cells showed on the blade and then the result is converted to %. Cells viability should be over 98%.

C) Experience Animals and Control Animals Grafting with 256 Walker Sarcoma (Solid Tumor), and with T8 Guerin Lympho-Trop Epitelioma (Solid Tumor)
In the 60^{th} day from the preliminary treatment with Deuterium depleted Water administered to the animals as per item A), the grafting of both rats to be tested and control rats is initiated using well known procedures, which means subcutaneous, dorsally, with 1×10⁷ malign tumor cells in 0.5 natural saline solution of 256 Walker sarcoma (solid tumor) and with T8 Guerin lympho-trop epitelioma (solid tumor), both of them having cells with a viability of over 98%.
The grafting has been performed on three groups of 220 animals each, these animals being fed with Deuterium depleted Water of three types of concentrations as: 25 ppm, 60 ppm and 100 ppm, respectively.
From each group of 220 tested animals, two groups of 79 animals were grafted with 256 Walker sarcoma (solid tumor) and another 79 animals was grafted with T8 Guerin lymphotrop epitelioma (solid tumor).
The other animals were used as control animals.

D) Continuously Administering and on Long Term, by Diet, of Deuterium Depleted Water, with Concentrations of 25 ppm, 60 ppm, 100 ppm Deuterium Compared to 150 ppm Deuterium Tap Water Administering to Control Animals
Tested animals, which were constituting three groups for testing, have been administering, by daily diet, Deuterium Depleted Water having three concentrations: 25 ppm; 60 ppm and 100 ppm, over a period of 700 days. The beginning of administering to the tested animals was simultaneous with the beginning of administering to control animals, as daily diet too, of tap water having a Deuterium content of 150 ppm.
During the experiment, the following has been done:
a. Starting with the 4^{th} day after grafting, the tumor nodules developed to tested animals are examinated and measured, on each 2-3 days, at the inoculation spot. Tumoral incidence has been of 100% over all the groups. The tumor growth has been assessed by measuring two kind of diameters: large diameter and small diameter on different stages of tumoral growth. Tumoral volume was calculated using the equation V=a×b²×0.4 where V is the volume measured in mm³; a and b are the two diameters and 0.4 is a constant.
b. Daily, the physiological condition of animals was inspected by weighing, food and water consumption monitoring and by observing the occurrence of toxic phenomena (bleedings, diarrhea, hair loss, etc.). Also, mortality rate was daily recorded. The animals were weighed at the beginning of the tumor measurements because their sudden loss of weight could have been an expression of toxic phenomena.
c. After 60 days, when all the control animals ceased to live, preferable between the 160 ^{th} day and the 200^{th} day after grafting date, the effect produced by Deuterium Depleted Water is observed, at stated concentration, on surviving tested animals homeostasis, that means how the humoral immune system and cellular immune system were influenced at these animals, by conducting a series of examinations of immunological state of animals, namely: leucocytes formula test to establish lymphocytes and blastic cells levels including the levels of Nk-K cells and dendritic cells presence; analyses on hematopoietic marrow on lympho-nodal areas that are satellite to tumor graft zone, including tumor grafted formation.

E) Determination of Efficient Concentration of Deuterium Depleted Water for Tested Surviving Animals Depending on New Homeostasis Occurrence, and on the Obtained Results-Related Tumor Regression and Cancer Curing.

The assessing of anti-tumoral activity of Deuterium Depleted Water was done as per NCI (1990) criteria. Tumor Growth Inhibition (TGI %) was measured and a value of 50% of this indicator showed a strong inhibition of tumoral growth.

For animal survival the followings were calculated:

Mean Time of Survival (MTS)
T/C ratio (Treated/Control×100) which must be bigger than 125;
Increase Life Survival (ILS %) that must have a value over 25-36, depending on experimental system used;
Counting the number of long term living animals versus treated animals number

Standard procedures were followed for GLP validation of the experimental models used in pharmaceutics surveys. The two variables required by GLP were met: independent (prediction) variable, which means the use of the same number of tumor cells inoculated in the same standard conditions on all animals (1×10⁷ tumor cells) and dependent (criterion) variable, which means:
latent period (expressed as days from tumoral transplant until the occurrence of a palpable tumor nodule);
tumoral incidence: the number of animals having tumors, number of animal without tumors, tumor rejections
Mean Time of Survival (days MTS) from the tumor graft until the death of the last animal.

### RESULTS

As a first result, we may stress the fact that there are two significant differences as regards tumor generation and tumor development of the control to which current tap water has been administered as a diet, before and after tumor graft and the rats to which Deuterium Depleted Water has been administered, especially of a 60 ppm concentration.

As regarding latent period, it has been demonstrated a prolongation of this period with 5 days at the groups having Deuterium Depleted Water on a 60 ppm concentration versus control group, which is true both for 256 Walker tumor and T8 Guerin tumor. This prolongation of latent period has influenced the percentage of rats with tumors in different stages of tumoral growth.

As regarding animal survival the followings have been demonstrated:
after 60 days from tumor inoculation a slight prolongation of MTS has been noted at the T8 Guérin tumor grafted animals to which 60 ppm concentration Deuterium Depleted Water was administered, and also, a significant MTS prolongation has been noted at the 256 Walker tumor grafted animals. At the 256 Walker tumor grafted animals the assessment criteria for tumoral activity indicated significant values too (T/C %=150 and ILS %=50);
at 256 Walker tumor grafted animals to which 60 ppm Deuterium Depleted Water was administered, in the 60^{th} day, 33/47 (41%) rats were still living versus 20% at the animals treated with 25 ppm Deuterium concentration water, and 28.5% at the animals treated with 100 ppm Deuterium water;
until the 60^{th} day from the tumor inoculation date, all the control animals died because of cancer;
at 80 days, out 79 256 Walker tumor grafted animals treated with 60 ppm Deuterium Depleted Water, 27 were still living, and 22 of these had no any tumor. The percentage for those rats not developing cancer was 34%.
as for T8 Guérin tumor and 60 ppm concentration Deuterium Depleted Water, at 184 days, there have been 8/77 rats (10.4%) not developing cancer, and 11/77 (14.2%) showed a very slow tumor growth;
at 584 days, for Walker tumor and 60 ppm concentration Deuterium Depleted Water, 20/70 rats were still living, which means 28.5%, and 8/60 (11.1%) rats showed a very much delayed tumor growth.

These results concluded after method application as per invention procedure, are due first of all, to cell modulation in immunity system that was humoral and cellular mediated, which determines an inhibition on development and growth of the two types of malign tumor experimented (256 Walker and T8 Guérin). The evidence of this action is illustrated by the followings:
on 162 days for 256 Walker tumor and on 192 days for T8 Guérin tumor, leucocytes formula at the surviving animals having an extremely slow tumor growth, showed an increased percentage of lymphocytes of about 70-80% versus 15-20% showed at the control;
recording of an increased percentage of dendritic cells (5-8% versus 0-1% at the control) and of NK-K cells (9-15% versus 0-1% at control);
it was concluded that hematopoietic marrow of the animals treated with 60 ppm Deuterium Depleted Water showed a massive infiltration of immunoblasts, plasmocytes and mastocytes, which demonstrated a specific cellularity for immune reaction humoral mediated
lymphopoietic territory showed images identical to the ones in hematopoietic marrow;
tumors cellularity having an extremely slow growth is quite different from the one existing at control. Thus, while at the control tumoral cells were in a proportion of approximately 100% from the cellularity, at the animals fed with Deuterium Depleted Water, especially the 60 ppm Deuterium Depleted Water, the threshold for these tumoral cells was not exceeding 10%, the remaining tumoral cells being in necrobiosis;
it is remarked the tumor invasion by lymphocytes and NK-K cells
cytomorphologic analyze of hematopoiety showed a leucocytes formula with a high level of lymphocytes (60-75%), blastic lymphoid cells (5-7%), the presence of dendritic cells (5-8%) and of NK-K cells (9-15%) at the rats that were exclusively and continuously consuming 60 ppm Deuterium Depleted Water over a period of 1-2 years.

Therefore, cancer-surviving rats showed a very long-term remanence of this extraordinary immunity stimulation by continuously administering of 60 ppm Deuterium Depleted Water before and after tumoral graft.

Based on the results gained by method application as per invention procedure, may conclude that continuously administering of 60 ppm Deuterium Depleted Water, over a period of 60 days before tumor graft and the administering of this kind of water over a prolonged period of time does inhibit the development and growth of the two malign tumors experienced on outbred Wistar rats, finally resulting in non-development of cancer in a proportion of a significant percentage, as well as the significant prolongation of survival time for the animals having tumors, being the cause of a growth retardation.

Also, we can state that Deuterium Depleted Water of 60 ppm concentration acts as a homeostasis factor for inhibition of malign tumor development and growth when it is continuously administered over a long period of time.

Since cancer-surviving rats showed a very long-term remanence of immune system after continuously administering of 60 ppm Deuterium Depleted Water, we can state that Deuterium Depleted Water of a 60 ppm concentration generated a new hemostasis at the tested animals to which it had been administered, producing a modulation of the cells in humoral and cellular mediated immunity system.

To maintain the homeostasis induced by Deuterium Depleted Water it is necessary that the administering of this water, once started, should be indefinitely continued without accidental interruptions, otherwise, the occurred homeostasis could be easily disturbed and the homeostasis stage prior to Deuterium Depleted Water could revert when homeostasis conditions facilitate again the proliferation of malign clone.

1-2. (canceled)

3. A method for in vivo determination on tested animals of the efficient concentration of deuterium depleted water for cancer therapy, wherein deuterium depleted water is provided for administering to tested animals before and after tumor grafting with animal grafts, the method comprising:
A) administering deuterium depleted water to Wistar outbred rats by diet, with concentration less than 100 ppm, over a period of 60 days, simultaneously to dieting a control group of animals with water having 150 ppm content of Deuterium (tap water), over the same period of time;
B) determining viability for the tumor cells to be grafted, using tripan blue;
C) grafting of the animals in the experimental group and the control animals in the 60th day, subcutaneous, with 1×107 malign tumor cells in 0,5 ml normal saline solution of 256 Walker sarcoma (the solid tumor) and T8 Guerin lymphotropic epitelioma (solid tumor), both of them having cells with a viability over 98%;
D) administering continuously and long-term, by diet, of deuterium depleted water, with concentration less than 100 ppm deuterium, period over which the followings are to be done:
a. starting with the 4-th post-graft day the tumor nodules measurement and examination is performed on each 2-3 days;
b. monitoring of animals physiological condition by weekly weighing, monitoring their food and water consumption, notifying the toxic condition occurrence;
c. after 60 days, when all the animals in control group are dead, preferably between the 160th and 200th day after graft, the effect produced by administering of established concentration of deuterium depleted water is observed on the surviving animals homeostasis from experimental groups, respectively the way how humoral immune system and cellular immune system of these animals has been influenced, by performing of a series of examination on immunological condition of the animals, namely: leucocytes formula test to establish lymphocytes and blastic cells levels; hematopoietic marrow tests to establish the plasmocytes and NK-K cells levels; and
E) determining of efficient concentration of deuterium depleted water for tested surviving animals depending on new homeostasis occurrence, and on the results obtained related to tumoral regression, as well as to cancer curing.

4. The method according to claim 3, wherein it is determined that the 60 ppm deuterium depleted water as the concentration that is the most efficient for cancer therapy and prophylaxis by continuously and long-term administering of this type of water as a daily diet.
